(19) United States
(12) Patent Application Publication
(10) Pub. No.: US 2005/0276788 A1
(43) Pub. Date: Dec. 15, 2005

(54) Self-containing lactobacillus strain

(75) Inventors: Lothar Steidler, Bandon (IE); Pieter Rottiers, De Pinte (BE); Erik Remaut, Vinderhoute (BE)

Correspondence Address: TRASK BRITT, P.O. BOX 2550, SALT LAKE CITY, UT, 84110, US

(73) Assignees: Vlaams Interuniversitair Instituut Voor Biotechnologie VZW, Zwijnaarde (BE); Universiteit Gent, Gent (BE)

(21) Appl. No.: 11/127,921

(22) Filed: May 12, 2005

Related U.S. Application Data
(63) Continuation of application No. PCT/EP04/46346, filed on Nov. 14, 2003.

(30) Foreign Application Priority Data
Nov. 15, 2002 (EP) 02079786.6

(51) Int. Cl. A61K 48/00; C12N 1/21

(52) U.S. Cl. 424/93.2; 435/252.3; 435/252.9

(57) ABSTRACT

The invention relates to a recombinant Lactobacillus strain, with limited growth and viability in the environment. More particularly, it relates to a recombinant Lactobacillus that can only survive in a medium, where well-defined medium compounds, preferably thymidine or thymine, are present. A preferred embodiment is a Lactobacillus that may only survive in a host organism, where the medium compounds are present, but cannot survive outside the host organism in absence of the medium compounds. Moreover, the Lactobacillus strain can be transformed with prophylactic and/or therapeutic molecules and can, as such, be used to treat diseases such as, but not limited to, inflammatory bowel diseases.

### CROSS-REFERENCE TO RELATED APPLICATIONS

This application is a continuation of PCT International Patent Application No. PCT/EP2004/046346, filed on Nov. 14, 2003, designating the United States of America, and published in English, as PCT International Publication No. WO 2004/046346 A2 on Jun. 3, 2004, which application claims priority to European Patent Application Serial No. 02079786.6, filed Nov. 15, 2002, the entirety of each of which being incorporated herein by this reference.

### TECHNICAL FIELD

The invention relates generally to biotechnology, and more particularly to a recombinant Lactobacillus strain with limited growth and viability in the environment. More specifically, it relates to a recombinant Lactobacillus that only survives in a medium where well-defined medium compounds, such as thymidine or thymine, are present. In one embodiment, a Lactobacillus that may only survive in a host organism where the medium compounds are present, but cannot survive outside the host organism in absence of the medium compounds. Moreover, the Lactobacillus strain can be transformed with prophylactic and/or therapeutic molecules and can, as such, be used to treat diseases such as, but not limited to, inflammatory bowel diseases.

### BACKGROUND

Lactic acid bacteria have long been used in a wide variety of industrial fermentation processes. They have “generally regarded as safe” status, making them potentially useful organisms for the production of commercially important proteins. Indeed, several heterologous proteins, such as Interleukin-2, have been successfully produced in Lactococcus spp (Steidler et al., 1995). It is, however, undesirable that such genetically modified microorganisms are surviving and spreading in the environment.

To avoid unintentional release of genetically modified microorganisms, special guidelines for safe handling and technical requirements for physical containment are used. Although this may be useful in industrial fermentations, the physical containment is generally considered as insufficient, and additional biological containment measures are taken to reduce the possibility of survival of the genetically modified microorganism in the environment. Biological containment is extremely important in cases where physical containment is difficult or even not applicable. This is, amongst others, the case in applications where genetically modified microorganisms are used as live vaccines or as vehicles for delivery of therapeutic compounds. Such applications have been described, for example, in PCT International Patent Publication WO 97/14806, that disclose the delivery of biologically active peptides, such as cytokines, to a subject by recombinant non-invasive or non-pathogenic bacteria. PCT International Patent Publication WO 96/11277 describes the delivery of therapeutic compounds to an animal, including humans, by administration of a recombinant bacterium encoding the therapeutic protein. Steidler et al. (2000) describe the treatment of colitis by administration of a recombinant Lactococcus lactis, secreting interleukin-10. Such a delivery may indeed be extremely useful to treat a disease in an affected human or animal, but the recombinant bacterium may act as a harmful and pathogenic microorganism when it enters a non-affected subject and an efficient biological containment that avoids such unintentional spreading of the microorganism is needed.

Although a sufficient treatment can be obtained using Lactococcus, it has as main disadvantage that the bacterium is not colonizing and that the medication should applied in a continuous way to ensure the effect. A colonizing strain like Lactobacillus would have the advantage that a similar effect can be used with a single dose or a limited number of doses. However, similar to the Lactobacillus case, a stringent biological containment system is needed to avoid the dissemination of the bacterium in the environment.

Biological containment systems for host organisms may be passive, based on a strict requirement of the host for specific growth factor or a nutrient that is not present or present in low concentrations in the outside environment, or active, based on so-called suicidal genetic elements in the host, wherein the host is killed in the outside environment by a cell-killing function, encoded by a gene that is under control of a promoter only being expressed under specific environmental conditions.

Passive biological containment systems are well known in microorganisms such as Escherichia coli or Saccharomyces cerevisiae. Such E. coli strains are disclosed, e.g., in U.S. Pat. No. 4,100,495. WO 95/10621 discloses lactic acid bacterial suppressor mutants and their use as means of containment in lactic acid bacteria, but in that case, the containment is on the level of the plasmid, rather than on the level of the host strain and it stabilizes the plasmid in the host strain, but does not provide containment for the genetically modified host strain itself A similar containment system on the level of the plasmid has been described for Lactobacillus acidophilus by Fu and Xu (2000), using the thyA gene from Lactobacillus casei as the selective marker. The thyA mutant used has been selected by spontaneous mutagenesis and trimethoprim selection. Such a mutation is prone to reversion and the thyA gene of another Lactobacillus species is used to avoid the reversion of the mutation by inrecombination of the marker gene. Indeed, reversion of the thyA mutation is a problem and, especially in absence of thymine or thymidine in the medium, the mutation will revert at high frequency, wherein the strain is losing its containment characteristics. For an acceptable biological containment, a non-reverting mutant is wanted.

Non-reverting mutants can be obtained by gene disruption. However, although the thyA gene of Lactobacillus casei has been mutated by site-directed mutagenesis, it was only tested in E. coli and never used for gene replacement in a Lactobacillus strain. Although transformation techniques for Lactobacillus are known to the person skilled in the art, gene disruption of thyA in Lactobacillus has never succeeded and is clearly not evident.

Active suicidal systems have been described by several authors. Such systems consist of two elements: a lethal gene and a control sequence that switches on the expression of the lethal gene under non-permissive conditions. WO 95/10614 discloses the use of a cytoplasmatically active truncated and/or mutated Staphylococcus aureus nuclease as the lethal gene. WO 96/40947 discloses a recombinant bacterial system with environmentally limited viability, based on the expression of either an essential gene, expressed when the cell is in the permissive environment and is not expressed or temporarily expressed when the cell is in the non-permissive environment and/or a lethal gene, wherein expression of the gene is lethal to the cell and the lethal gene is expressed when the cell is in the non-permissive environment but not when the cell is in the permissive environment. WO 99/58652 describes a biological containment system based on the relE cytotoxin. However, most systems have been elaborated for E. coli (Tedin et al., 1995; Knudsen et al., 1995; Schweder et al., 1995) or for Pseudomonas (Kaplan et al., 1999; Molina et al., 1998). Although several of the containment systems theoretically can be applied to lactic acid bacteria, no specific biological containment system for Lactobacillus has been described that allows the usage of a self-containing and transformed Lactobacillus to deliver prophylactic and/or therapeutic molecules in order to prevent and/or treat diseases.

### SUMMARY OF THE INVENTION

In one embodiment, the present invention provides a suitable biological containment system for Lactobacillus.

A first aspect of the invention is an isolated strain of Lactobacillus sp. comprising a mutant thymidylate synthase gene (thyA), wherein the gene is inactivated by gene disruption. “Gene disruption,” as used herein, includes disruption insertion of a DNA fragment, disruption by deletion of the gene, or a part thereof, as well as exchange of the gene or a part thereof by another DNA fragment. Preferably, disruption is the exchange of the gene, or a part thereof, by another functional gene. Preferably, the mutant thymidylate synthase is a non-reverting mutant.

A “non-reverting mutant,” as used herein, means that the reversion frequency is lower than 10⁻⁸, preferably the reversion frequency is lower than 10⁻¹⁰, even more preferably, the reversion frequency is lower than 10⁻¹², even more preferably, the reversion frequency is lower than 10⁻¹⁴, most preferably, the reversion frequency is not detectable using the routine methods known to the person skilled in the art. Preferably, Lactobacillus sp. is L. salivarius or L. plantarum. A non-reverting thyA mutant strain can be considered as a form of active containment as it will undergo cell death in response to thymine and thymidine starvation (Ahmad et al., 1998).

The L. casei thymidylate synthase gene has been cloned by Pinter et al. (1988). CN1182134 discloses a vector devoid of antibiotic resistance and bearing a thymidylate synthase gene as a selection marker; the same vector has been described by Fu and Xu (2000) for L. acidophilus. However, in this specific case, reversion of the mutation is prevented by complementing the mutation by the L. casei gene that shows only a low homology; the stability of the mutation is only guaranteed in the presence of the complementing vector or when thymine or thymidine is supplied to the medium. The mutant strain may not be stable enough to use in medical situations where a strict biological containment is needed. Disclosed herein is how to construct such a mutant by gene disruption, using homologous recombination in Lactobacillus.

In one embodiment, the thyA gene of a Lactobacillus sp. strain, preferably L. salivarius or L. plantarum, is disrupted and replaced by a functional human interleukin-10 expression cassette. The interleukin-10 expression unit is preferably, but not limited to, a human interleukin-10 expression unit or gene encoding for human interleukin-10. However, it is clear that any construct can be used for gene disruption, as long as it results in an inactivation of the thyA gene or in an inactive thymidylate synthase. As a non-limiting example, the homologous recombination may result in a deletion of the gene, in one or more amino acid substitutions that lead to an inactive form of the thymidylate synthase, or to a frameshift mutation resulting in a truncated form of the protein.

Another aspect of the invention is the use of a strain according to the invention as host strain for transformation, wherein the transforming plasmid does not comprise an intact thymidylate synthase gene. Such a Lactobacillus sp. thyA mutant is very useful as a host strain in situations where more severe containment than purely physical containment is needed. Indeed, thyA mutants cannot survive in an environment without, or with only a limited concentration of, thymidine and/or thymine. When such a strain is transformed with a plasmid that does not comprise an intact thyA gene and cannot complement the mutation, the transformed strain will become suicidal in a thymidine/thymine-poor environment. Such a strain can be used in a fermentor as an additional protection for the physical containment. Moreover, the present invention discloses that such a strain is especially useful in cases where the strain is used as a delivery vehicle in an animal body, including the human body. Indeed, when such a transformed strain is given, for example, orally to an animal, including humans, it survives in the gut and produces homologous and/or heterologous proteins, such as human interleukin-10, that may be beneficial for the animal.

Still another aspect of the invention is a transformed strain of Lactobacillus sp. according to the invention comprising a plasmid that does not comprise an intact thymidylate synthase gene. The transforming plasmid can be any plasmid, as long as it cannot complement the thyA mutation. It may be a self-replicating plasmid that preferably carries one or more genes of interest and one or more resistance markers or it may be an integrative plasmid. In the latter case, a special case of transformation is the one wherein the integrative plasmid itself is used to create the thyA mutation by causing integration at the thyA site, wherein the thyA gene is inactivated. Preferably, the active thyA gene is replaced by double homologous recombination by a cassette comprising the gene or genes of interest, flanked by targeting sequences that target the insertion to the thyA target site. In this case, the introduction of the mutation and the transformation with the gene of interest is carried out in one and the same transformation experiment. It is of extreme importance that these targeting sequences are sufficiently long and sufficiently homologous to obtain integration of the sequence into the target site. However, to avoid the problem of the long homologous sequences, a recombinase-assisted cross-over may be used. Transformation methods of Lactobacillus are known to the person skilled in the art and include, but are not limited to, protoplast transformation and electroporation.

Another aspect of the invention relates to a transformed strain of Lactobacillus sp. comprising a gene or expression unit encoding a prophylactic and/or therapeutic molecule. Preferably, the prophylactic and/or therapeutic molecule is interleukin-10.

Consequently, the present invention also relates to the usage of a transformed strain of Lactobacillus sp. to deliver prophylactic and/or therapeutic molecules and, as such, to treat diseases. The delivery of such molecules has been disclosed as a non-limiting example in WO 97/14806 and in WO 98/31786. Prophylactic and/or therapeutic molecules include, but are not limited to, polypeptides such as insulin, growth hormone, prolactine, calcitonin, group 1 cytokines, group 2 cytokines and group 3 cytokines and polysaccharides such as polysaccharide antigens from pathogenic bacteria. A preferred embodiment is the use of a Lactobacillus sp. strain according to the invention to deliver human interleukin-10. Methods to deliver the molecules and methods to treat diseases such as inflammatory bowel diseases are explained in detail in WO 97/14806 and WO 00/23471 to Steidler et al. and in Steidler et al. (2000) that are hereby incorporated by reference. The present invention demonstrates that the strain according to the invention surprisingly passes the gut at the same speed as the control strains and shows that their loss of viability is indeed not different from that of the control strains. However, once the strain is secreted in the environment, for example, in the feces, it is not able to survive any longer. The fact that the deletion mutant can survive in the intestine, and more specifically in the ileum, and as such, can be used as a biologically contained delivery strain, is especially surprising, as it is known that the dependency upon thymine by the known thyA mutants is rather high (about 20 μg/ml; Ahmad et al., 1998). Based on this data, one would expect that mutant cannot survive in the ileum where there is only a very limited concentration of thymine present.

Another aspect of the invention is a pharmaceutical composition comprising a Lactobacillus sp. thyA disruption mutant according to the invention. As a non-limiting example, the bacteria may be encapsulated to improve the delivery to the intestine. Methods for encapsulation are known to the person skilled in the art and are disclosed, amongst others, in EP 0450176.

Still another aspect of the invention is the use of a strain according to the invention for the preparation of a medicament. Preferably, the medicament is used to treat Crohn's disease or inflammatory bowel disease.

### DESCRIPTION OF THE FIGURES

FIG. 1: plasmid map of the pKD46 plasmid that, upon arabinose induction, expresses the phage λ Red recombinases. Bla, ampicillin resistance; gam, γ gene; bet, β gene; exo, exo gene; P_{araB}, arabinose-inducible promoter.

FIG. 2: Plasmid map of ORI⁺ RepA⁻ pORI19. LacZ, lacZα fragment from pUC19. Em, erythromycin resistance gene. Only relevant restriction enzyme sites are shown.

FIG. 3: Construction schedule of the vector pORI-RED.

FIG. 4: System of gene-replacement of the Lactobacillus thyA gene by hIL-10 with the aid of the lambda red recombinases.

### DETAILED DESCRIPTION OF THE INVENTION

### EXAMPLES

### Example 1

### General Outline of the Experiment

On the base of the Lactobacillus casei or the Lactobacillus plantarum sequence, the Thy A gene is localized in L. salivarius, or any other suitable Lactobacillus species. Starting from this sequence, the sequences adjacent to the Thy A gene are cloned and sequenced.

The knowledge of these sequences is of critical importance for the genetic engineering of any Lactobacillus strain in a way as described below, as the strategy will employ double homologous recombination in the areas 1000 bp at the 5′ end and 1000 bp at the 3′ end of thyA, the “thyA target.” These sequences are not available from any public source to date. We have cloned these flanking DNA fragments and have identified their sequence.

The thyA replacement is performed by homologous recombination, essentially as described by Biwas et al. (1993). Suitable replacements in a plasmid-borne version of the thyA target are made, as described below. The carrier plasmid is a replication-defective plasmid, which only transfers the erythromycin resistance to a given strain when a first homologous recombination occurs at either the 5′ 1000 bp or at the 3′ 1000 bp of the thyA target. A second homologous recombination at the 3′ 1000 bp or at the 5′ 1000 bp of the thyA target yields the desired strain. Alternatively, a recombinase-assisted inrecombination may be used. This allows the use of shorter 5′ and 3′ sequences.

The thyA gene is replaced by a synthetic gene encoding a protein that has a secretion leader, functional in Lactobacillus, fused to a protein of identical amino acid sequence than: (a) the mature part of human-interleukin 10 (hIL-10) or (b) the mature part of hIL-10 in which proline at position 2 had been replaced with alanine.

The resulting strains are thyA deficient, a mutant not yet described for L. salivarius. It is strictly dependent upon the addition of thymine or thymidine for growth.

The region around the inserted hIL-10 gene is isolated by PCR and the DNA sequence is verified. The structure is identical to the predicted sequence.

Human interleukin-10 production in the mutants is checked by Western blot analysis and compared with the parental strain, transformed with an empty plasmid as negative control, and the parental strain, transformed with the IL-10-producing plasmid as positive control. The concentration in the culture supernatant is quantified using ELISA. All isolates of the mutant produce a comparable, significant amount of hIL-10, be it less than the strain, transformed with the non-integrative plasmid.

Quantification of hIL-10 present in the culture supernatant of the indicated strains is done by ELISA. The N-terminal protein sequence of the recombinant hIL-10 is determined by Edman degradation and is shown identical to the structure as predicted for the mature, recombinant hIL-10. The protein shows full biological activity.

The effect of the thymidilate synthase deletion on the growth in thymidine-less and thymidine-supplemented media is tested. Absence of thymidine in the medium strongly limits the growth of the mutant and even results in a decrease of colony-forming units after four hours of cultivation in absence of thymidine or thymine. Addition of thymidine to the medium results in an identical growth curve and amount of colony-forming units, compared to the wild-type strain, indicating that the mutant does not affect the growth or viability in thymidine-supplemented medium.

Mouse experiments are carried out, proving that the L. salivarius thyA mutant is able to survive in the ileum of the mice, but cannot survive outside the intestine. The colony count of the mutant in the feces drops dramatically, when compared to the wild type strain, indicating that the strain is a useful tool for delivery in the intestine under conditions of biological containment.

### Example 2

### Identification of the Thymidylate Synthase (thyA) Regio in Lactobacillus Species

Based on the publication of Kleerebezem et al., 2003, we had web-based access to the complete genome sequence of Lactobacillus plantarum WCFS1. Based on a blastn between the complete genome of the Lactobacillus plantarum WCFS1 and the thyA gene of E. coli K12, we identified the thyA gene in Lactobacillus.

Based on these published thyA DNA sequences of L. plantarum, WCFS1 degenerate oligonucleotides are synthesized to be used as primers for DNA sequencing of the thyA gene of any particular Lactobacillus species. Once the sequence of the thyA gene of that particular Lactobacillus species is known, oligonucleotides are designed as primers for DNA sequencing of the 5′ and 3′ flanking regions of the thyA gene. The identification of the 5′ and 3′ flanking regions (a stretch of 50 nucleotides upstream and downstream of the thyA gene is sufficient) is necessary for the gene replacement of the thyA gene by the human interleukin-10 gene (hIL-10 gene).

### Example 3

### Gene-Replacement of the thyA Gene by the hIL-10 Gene

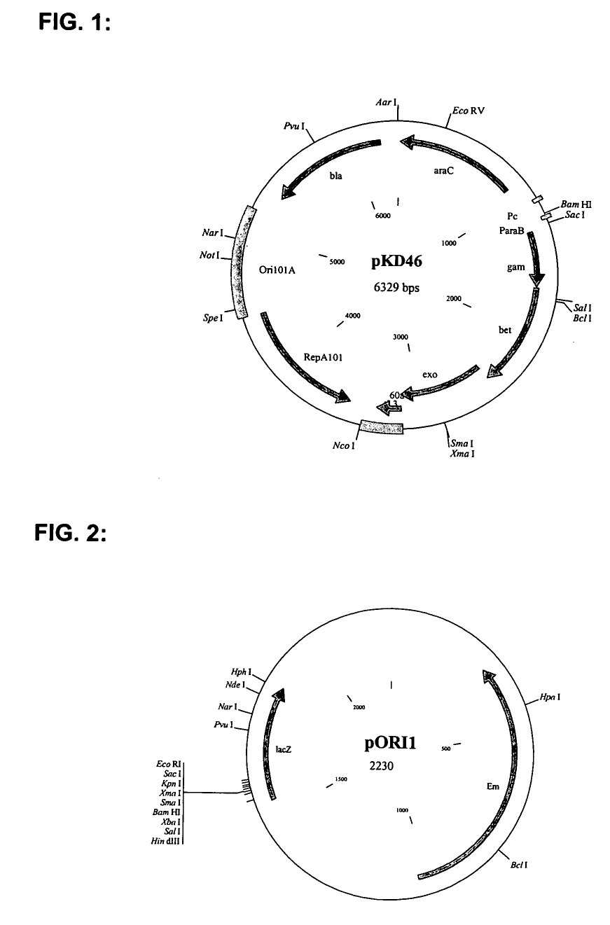

The system of gene-replacement that is used in Lactobacillus is an adaptation of a system introduced by Datsenko et al. (2000). This is a simple and highly efficient method to disrupt chromosomal genes in Escherichia coli. In this procedure, PCR primers provide the homology to the targeted gene(s) and recombination depends on the phage λ Red recombinases, which are synthesized under the control of an arabinose-inducible promoter on an easily curable, low copy number plasmid, plasmid pKD46 (FIG. 1). This recombination pathway not only ensures that, after electroporation of the linear PCR fragment into the cell, the linear DNA is not instantly degraded, but it also allows an efficient gene replacement by a double cross-over with a limited homology of only 36 to 50 nucleotides to the regions adjacent to the gene that need to be replaced.

The pKD46 plasmid is an E. coli plasmid. To adapt this method to Lactobacillus, it is necessary that the λ Red recombinases are subcloned into a plasmid that can replicate in Lactobacillus. The λ Red recombinase operon is subcloned in the broad host shuttle vector pORI19 (FIG. 2; Law et al., 1995). pORI19 is preferred because it is based on the conditional replicon of the lactococcal pWV01-derived Ori⁺ RepA⁻vector. Due to the fact that the pORI19 is missing the repA gene, it is replication deficient. For the replication of the pORI19 plasmid, the helper plasmid pVE6007 (Maguin et al., 1992) needs to provide the RepA-Ts protein in trans. The replication of the helper plasmid pVE6007 is temperature sensitive. A temperature of 30° C. is permissive for the replication of the plasmid, while a temperature shift to 37° C. abolishes its replication and induces the loss of the plasmid. The loss of the helper plasmid pVE6007 results in the loss of the pORI19 plasmid. Assembly of pORI19-derived plasmids is carried out in the E. coli helper strain EC101, which has the repA gene genomically integrated.

Construction of pORI-RED

pORI-RED is the pORI19 plasmid in which the λ Red recombinase operon from the vector pKD46 is subcloned under control of the arabinose inducible promotor. All the constructs are made in the E. coli helper strain EC 101.

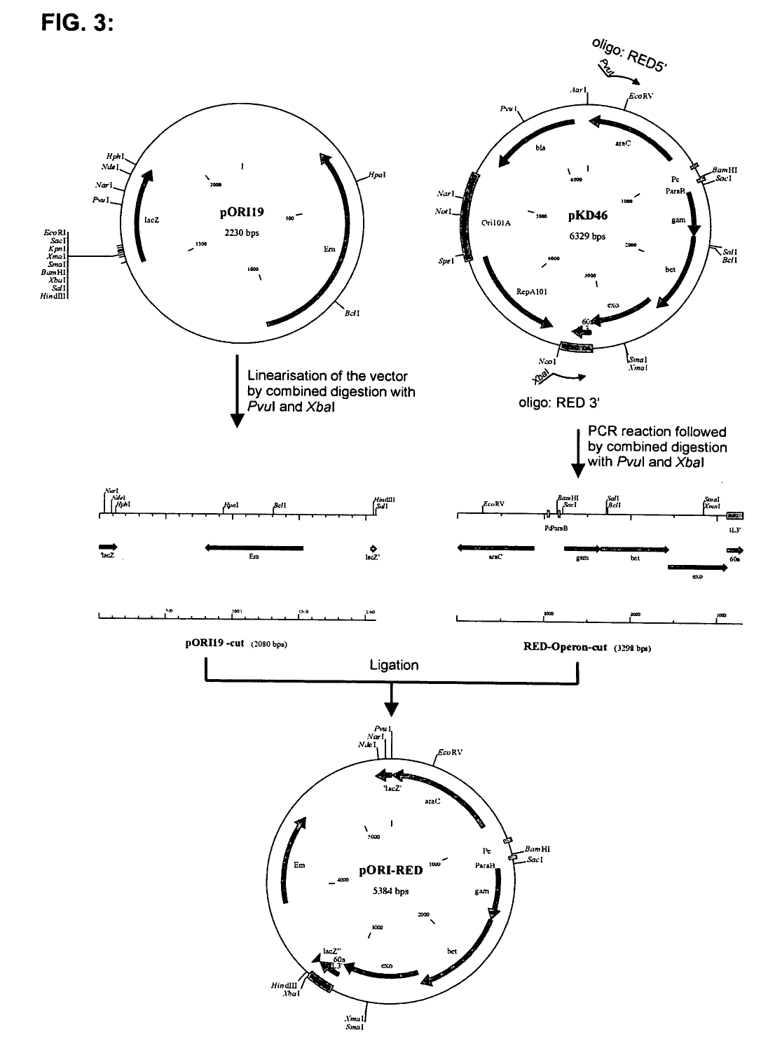

By use of PCR, the λ Red recombinase operon is amplified (FIG. 3). The primers of the PCR are designed in such a way that a PvuI site is introduced at the 5′ end of the operon and an XbaI site is introduced at the 3′ end. This PCR fragment is cut by a combined digestion of PvuI and XbaI and ligated in by the PvuI and XbaI linearized pORI19 vector. This ligated plasmid is electroporated to the E. coli helper strain EC101 (for construction scheme, FIG. 3).

Preparation of the Recombination-Ready Lactobacillus Cells

Prior to gene replacement of the thyA gene by hIL-10, we prepare competent cells of the Lactobacillus strain and introduce the plasmids pVE6007 and pORI-RED by electroporation. Because of the temperature sensitivity of the plasmid pVE6007, all manipulations are conducted at 30° C. The introduction of these two plasmids in the Lactobacillus species is done in two steps. In the first step, the plasmid pVE6007 is electroporated in the electrocompetent Lactobacillus strain. Chloramphenicol is added to the medium to ensure the stability of pVE6007. The resulting Lactobacillus strain is made electrocompetent again and the plasmid pORI-RED is electroporated in this Lactobacillus strain, using erythromycin as the selectable marker. The resulting Lactobacillus strain harboring pVE6007 and pORI-RED is made electrocompetent by an adapted protocol. Thereto, an overnight Lactobacillus culture is 1/100 diluted in 250 ml MRS (Difco)+erythromycin and chloramphenicol, and 1 mM L-arabinose added. This ensures that the arabinose promotor of the pORI-RED plasmid is activated and that the three λ Red recombinases are expressed which makes recombination possible in the next step.

Generation of the Gene Replacement PCR Fragment

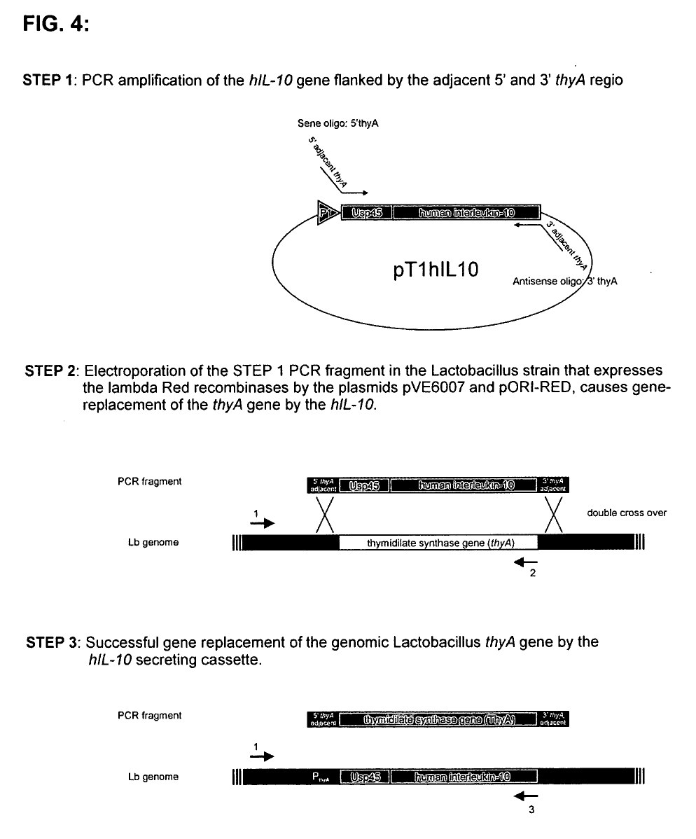

As described in FIG. 4, a linear PCR fragment is used for the gene replacement of the genomic thyA gene by the hIL-10 gene. For the PCR reaction, primers with 36- to 50-nucleotide extensions homologous to regions adjacent to the genomic thyA gene are used and a plasmid that carries the hIL-10 is used as template. This PCR was carried out on the template plasmid pTlhIL10 with the sense primer 5′ thyA and the antisense primer 3′ thyA (FIG. 4, STEP 1). The resulting PCR product is cleaned up with the Qiagen Qiaquick PCR purification kit (cat. # 28104). This purified PCR product is digested by DpnI for one hour to remove residual template (the plasmid pTlhIL10). Afterwards, the PCR product is fenol/chloroform extracted and precipitated by ethanol with the aid of see DNA (Amersham biotech, cat. # RPN 5200). The resulting PCR product pellet is dissolved in 5 μl TE buffer (Tris-EDTA).

Electroporation of the PCR Fragment into Lactobacillus

The PCR fragment that was generated in STEP 1, together with a selection plasmid, are now electroporated in the electrocompetent Lactobacillus strain containing the plasmids pVE6007 and pORI-RED. The 5 μl PCR mixture and the selection plasmid are mixed with 100 μl electrocompetent Lactobacillus cells. The cells are electroporated with a Biorad GENEPULSER™ II using the following conditions: 50 μF, 1.7 kV, 200 Ω whereafter 1 ml MRS+50 μg/ml thymidine is added to the cells. This Lactobacillus cell mixture is kept for two hours at 37° C. These two hours allow gene replacement of the genomic Lactobacillus thyA gene by the hIL-10 gene with the aid of the λ Red recombinases. By growing the cells at 37° C., the plasmid pVE6007 is inhibited in its replication and is lost, resulting in the subsequent loss of pORI-Red. After the two hours of incubation at 37° C., the Lactobacillus suspension is plated out at 30° C. on three MRS plates (350 μl per plate) containing 50 μg/ml thymidine and the antibiotic for which the selection plasmid specifies resistance. This step eliminates those cells in the electroporation mixture that were not competent for DNA uptake and provides a considerable enrichment for progeny cells derived from the fraction of competent cells that have taken up the selection plasmid. These have a high probability of also having taken up the linear PCR fragment generated in STEP 1.

### Example 4

### Identification of a thyA⁻ and IL-10⁺ Lactobacillus Primary thyA⁻ and IL-10+selection by PCR

The primary screening of the Lactobacillus colonies carrying a hIL-10 insert is done by colony PCR screening. A small part of each Lactobacillus colony is added to the respective PCR master mix. Two different PCR screenings are conducted on each Lactobacillus colony. The first PCR screening is the one where the primers are indicated by 1 and 2 on FIG. 4, STEP 2. In the negative colonies (no PCR product), the thyA gene is removed from the Lactobacillus genome and Lactobacillus strain is thyA negative. The second PCR screening is one with the primers 1 and 3 on FIG. 4, STEP 3. Positive colonies (a PCR product of approximately 1000 bp) are isolated. In these colonies, the Lactobacillus strain carries a genomically integrated copy of the hIL-10 gene. Confirmation of the thyA⁻ and IL-10+properties of the Lactobacillus by Southern blot

From the positive Lactobacillus colonies, a genomic DNA preparation is made. The genomic Lactobacillus DNA is digested by SpeI and NdeI and Southern blotted. The blot is revealed with digoxygenin-labeled probes for identifying thyA (thyA probe) or hIL-10 (hIL-10 probe). As expected based on the PCR results, the thyA probe signal is negative and the hIL-10 probe signal on the blot is positive.

### Example 5

### Production of Human IL-10 by the thyA⁻ and IL-10⁺ Lactobacillus

To evaluate the hIL-10 secretion, the strain is grown in buffered minimal medium (BM9) that contains 50 μg/ml thymidine. After 12 hours of growth at 37° C. of 4×10⁷ cells, the medium is tested for the prevalence of human IL-10 by Western blot and ELISA. The Lactobacillus strain is secreting a sufficient amount of human IL-10 in the culture supernatant to be used in in vivo experiments.

### Example 6

### Curing of Resident Plasmids

For use in in vivo experiments, the thyA⁻ and IL-10⁺ Lactobacillus strain is preferably free of any resident plasmid. This can be accomplished by successive rounds of curing (reviewed in: de Vos, 1987).

### REFERENCES

Ahmad, S. I., S. H. Kirk and A. Eisenstark (1998) Thymine metabolism and thymineless death in prokaryotes and eukaryotes. Annu. Rev. Microbiol. 52:591-625.
Biswas, I., A. Gruss, S. D. Ehrlich, et al. (1993) High-efficiency gene inactivation and replacement system for gram-positive bacteria. J. Bacteriol. 175:3628-3635.
De Vos, W. M. (1987) Gene cloning and expression in lactic streptococci. GEMS Microbiol. Rev. 46:281-295.
Datsenko K. A. and B. L. Wanner (2000) One-step inactivation of chromosomal genes in Escherichia coli K-12 using PCR products. Proc. Natl. Acad. Sci. U.S.A. 97:6640-6645.
Fu, X. and J. G. Xu (2000) Development of a chromosome-plasmid balanced lethal system for Lactobacillus acidophilus with ThyA gene as selective marker.
Gasson, M. J. (1983) Plasmid complements of Streptococcus lactis NCDO 712 and other lactic streptococci after protoplast-induced curing. J. Bacteriol. 154:1-9.
Kaplan, D. L., C. Mello, T. Sano, C. Cantor and C. Smith (1999) Streptavadin-based containment system for genetically engineered microorganisms. Biomol. Eng. 31:135-140.
Kleerebezem M., J. Boekhorst, R. van Kranenburg, D. Molenaar, O. P. Kuipers, R. Leer, R. Tarchini, S. A. Peters, H. M. Sandbrink, M. W. Fiers, W. Stiekema, R. M. Lankhorst, P. A. Bron, S. M. Hoffer, M. N. Groot, R. Kerkhoven, M. de Vries, B. Ursing, W. M. de Vos and R. J. Siezen (2003) Complete genome sequence of Lactobacillus plantarum WCFS1. Proc. Natl. Acad. Sci. U.S.A. 100:1990-1995.
Knudsen, S., P. Saadbye, L. H. Hansen, A. Collier, B. L. Jacobsen, J. Schlundt and O. H. Karlstrom (1995) Development and testing of improved suicide functions for biological containment of bacteria. Appl. Environ. Microbiol. 61:985-991.
Law J., G. Buist, A. Haandrikman, J. Kok, G. Venema and K. Leenhouts (1995) A system to generate chromosomal mutations in Lactococcus lactis which allows fast analysis of targeted genes. J. Bacteriol. 177:7011-7018.
Molina, L., C. Ramos, M. C. Ronchel, S. Molin and J. L. Ramos (1998) Construction of an efficient biologically contained pseudomonas putida strain and its survival in outdoor assays. Appl. Environ. Microbiol. 64:2072-2078.
Pinter, K., V. J. Davisson and D. V. Santi (1988) Cloning, sequencing, and expression of the Lactobacillus casei thymydilate synthase. DNA 7:235-241.
Schweder, T., K. Hofmann and M. Hecker (1995) Escherichia coli K12 relA strains as safe hosts for expression of recombinant DNA. Appl. Environ. Microbiol. 42:718-723.
Steidler, L., W. Hans, L. Schoffe, S. Neirynck, F. Obermeier, W. Falk, W. Fier and E. Remaut (2000) Treatment of murine colitis by Lactococcus lactis secreting Interleukin-10. Science 289:1352-1355.
Steidler, L., J. M. Wells, A. Raeymaekers, J. Vandekerckhove, W. Fiers and E. Remaut (1995) Secretion of biologically active murine Interleukin-2 by Lactococcus lactis subsp. Lactis. Appl. Environ. Microbiol. 61:1627-1629.
Tedin, K. A. Witte, G. Reisinger, W. Lubitz and U. Basi (1995) Evaluation of the E. coli ribosomal rrnB P1 promoter and phage derived lysis genes for the use in biological containment system: a concept study. J. Biotechnol. 39:137-148.

1. An isolated strain of Lactobacillus sp. carrying a mutant thyA gene, wherein said mutant thyA gene is inactivated by gene disruption.

2. The isolated strain of Lactobacillus sp. of claim 1, wherein said Lactobacillus sp. is Lactobacillus salivarius.

3. The isolated strain of Lactobacillus sp. of claim 1, wherein said Lactobacillus sp. is Lactobacillus plantarum.

4. The isolated strain of Lactobacillus sp. of claim 1, comprising a transforming plasmid that does not comprise an intact thymidylate synthase gene.

5. The isolated strain of Lactobacillus sp. of claim 1, further comprising a nucleotide sequence encoding a prophylactic and/or therapeutic molecule.

6. The isolated strain of Lactobacillus sp. of claim 5, wherein said prophylactic and/or therapeutic molecule is interleukin-10.

7. An improvement in a method of delivering a prophylactic and/or therapeutic molecule to a subject, the improvement comprising:
using the isolated strain of Lactobacillus sp. of claim 5 for the delivery of the prophylactic and/or therapeutic molecules to the subject.

8. A pharmaceutical composition comprising the isolated strain of Lactobacillus sp. of claim 5 presented in a pharmaceutically acceptable form.

9. A method of treating an inflammatory bowel disease in a subject, said method comprising administering, to the subject, the isolated strain of Lactobacillus sp. of claim 5 so as to treat the subject's inflammatory bowel disease.

10. A host strain for transformation comprising:
an isolated strain of Lactobacillus sp. carrying a mutant thyA gene, wherein said mutant thyA gene is inactivated by gene disruption and wherein a transforming plasmid contained therein does not comprise an intact thymidylate synthase gene.

11. The host strain for transformation of claim 10, wherein said Lactobacillus sp. is Lactobacillus salivarius.

12. The host strain for transformation of claim 10, wherein said Lactobacillus sp. is Lactobacillus plantarum.

13. The isolated strain of Lactobacillus sp. of claim 2, further comprising a nucleotide sequence encoding a prophylactic and/or therapeutic molecule.

14. The isolated strain of Lactobacillus sp. of claim 13, wherein said prophylactic and/or therapeutic molecule is interleukin-10.

15. The isolated strain of Lactobacillus sp. of claim 3 further comprising a nucleotide sequence encoding a prophylactic and/or therapeutic molecule.

16. The isolated strain of Lactobacillus sp. of claim 15, wherein said prophylactic and/or therapeutic molecule is interleukin-10.

17. The isolated strain of Lactobacillus sp. of claim 4, further comprising a nucleotide sequence encoding a prophylactic and/or therapeutic molecule.

18. The isolated strain of Lactobacillus sp. of claim 17, wherein said prophylactic and/or therapeutic molecule is interleukin-10.
